(19) United States
(12) Patent Application Publication
(10) Pub. No.: US 2014/0261421 A1
(43) Pub. Date: Sep. 18, 2014

(54) GAS FLOW DETECTOR AND POSITIVE AIRWAY PRESSURE APPARATUS CONTAINING THE SAME

(71) Applicant: APEX MEDICAL CORP., New Taipei City (TW)

(72) Inventor: Chia-Hsiang HSIAO, New Taipei City (TW)

(21) Appl. No.: 13/842,902

(22) Filed: Mar. 15, 2013

(51) Int. Cl. A61M 16/00; G01F 1/37

(52) U.S. Cl. CPC A61M 16/00; G01F 1/37
USPC 128/204.21; 73/861.47

(57) ABSTRACT

The disclosure provides a gas flow detector comprising a conduit, a first and a second pressure gauges. The conduit includes a flexible pipe, a first and a second connecting pipes. The flexible pipe has a contraction portion, a throat and an expanding portion. Further, the throat is sandwiched between the contraction portion and the expanding portion, and the inner diameter of the throat is smaller than that of the contraction portion and the expanding portion. The first and the second pressure gauges are configured to respectively detect the gas pressure of the first connecting pipe and the throat, and the gas flow rates of the first connecting pipe and the throat is calculated from the reading of the difference of the gas pressures. A positive airway pressure apparatus (PAPA) containing the gas flow detector is also disclosed herein.

### BACKGROUND

1. Technical Field

The present disclosure relates to a gas flow detector, and more particularly, to a gas flow detector used in a positive airway pressure apparatus (PAPA).

2. Description of Related Art

Obstructive sleep apnea (OSA) occurs when the muscles in the back of people's throat relax, resulting in difficulty in breathing or blockage in the airway. The OSA may cause snoring or upper airway resistance syndrome (UARS), and may even cause deadly intermittent apnea for patients in sleeping time.

FIG. 1A shows a normal breathing condition of a person in sleeping, in which the upper airway 11a is free open. FIG. 1B shows that, when the muscles in the back of the person's throat relax, the upper airway 11b becomes increasingly narrow, so as to cause snoring or upper airway resistance syndrome (UARS). It is shown in FIG. 1C that, when the muscles in the back of the person's throat relaxseverely, the person's upper airway 11c is completely blocked, and the OSA occurs accordingly.

In the study, it is found that there is 10% of population in the world suffering from the OSA, but only few of them are under proper treatments. For the patients, the OSA not only is life-threatening, but also may increases the risk of chronic diseases like hypertension or heart disease due to the bad sleeping quality.

FIG. 2 illustrates a diagram using a conventional positive airway pressure apparatus (PAPA) to treat the OSA, which is the most common treatment method at present. In the method, a positive pressure gas flow 21 is introduced into the upper airway 23 of a patient through a respirator 22, and the upper airway 23 of the patient is thus kept opened.

Generally, the conventional PAPA includes a gas flow detector having a conduit. The gas flow detector is used to regulate the gas flow rate introduced into the conduit by detecting the gas pressure in the conduit, in which the conventional conduit is made of rigid materials.

However, when the rigid materials are used in the conduit to detect gas pressure, the slope of gas pressure to gas flow rate is so large that it exceeds the detection limitation (2.0 hPa) of gas pressure of a detector at high gas flow rate (>140 LPM), for increasing the resolution at low gas flow rate (20-80 LPM), as shown in FIG. 3. Currently, the aforementioned problem is usually addressed by applying a special detector to expand the detecting scope, which significantly increases the cost of the product. Other method is proposed to decrease the resolution of gas flow rate so as to obtain a larger detecting scope, which however compromises the treating effect for the patients. Therefore, there is a need for an improved gas flow detector to solve the problems met in the art.

### SUMMARY

The present disclosure provides a gas flow detector and a positive airway pressure apparatus containing the same, so as to solve the problems of the prior art and increase the resolution at low gas flow rate.

One embodiment of the present disclosure is to provide a gas flow detector. The gas flow detector comprises a conduit, a first pressure gauge and a second pressure gauge.

The conduit comprises a flexible pipe, a first connecting pipe and a second connecting pipe. The flexible pipe comprises a contraction portion, a throat and an expanding portion. In which, the throat is sandwiched between the contraction portion and the expanding portion, and the inner diameter of the throat is smaller than the inner diameters of the contraction portion and the expanding portion. The first connecting pipe and the second connecting pipe are respectively connected to the contraction portion and the expanding portion of the flexible pipe.

The first pressure gauge is configured to measure the gas pressure of the first connecting pipe, and the second pressure gauge is configured to measure the gas pressure of the throat of the flexible pipe. The gas flow rates of the first connecting pipe and the throat of the flexible pipe are calculated based on the difference of gas pressures read from the first pressure gauge and the second pressure gauge.

Another embodiment of the present disclosure is to provide a positive airway pressure apparatus (PAPA). The PAPA comprises a flow generator, the said gas flow detector, a respirator and a control circuit.

The flow generator is configured to generate a positive pressure gas flow. The first connecting pipe of the conduit of the gas flow detector is connected to the flow generator, so as to introduce the positive pressure gas flow into the flexible pipe. The respirator is connected to the second connecting pipe of the conduit of the gas flow detector, such that the positive pressure gas flow is able to be introduced into the airway of a person under treatment and the airway is kept in positive pressure. The control circuit is electrically connected to the flow generator, and to the first pressure gauge and the second pressure gauge of the gas flow detector. The positive pressure gas flow of the flow generator is regulated by the pressure values read from the first pressure gauge and the second pressure gauge.

According to one example of the present disclosure, the Shore hardness of the flexible pipe is in a range of HS10-HS90.

According to one example of the present disclosure, the material of the flexible pipe is an elastomer comprising silicone, rubber, polyurethane, latex or polytetrafluoroethylene (PTFE).

According to one example of the present disclosure, the material of the first connecting pipe, the second connecting pipe or the both comprise flexible material or rigid material.

According to one example of the present disclosure, the material of the first connecting pipe, the second connecting pipe or the both are same as that of the flexible pipe.

According to one example of the present disclosure, the gas flow detector further comprises a honeycomb structure positioned in the throat of the flexible pipe, wherein the throat has a cross-section in a honeycomb shape.

According to one example of the present disclosure, the first pressure gauge is positioned on the inner wall of the first connecting pipe.

According to one example of the present disclosure, the pipe wall of the first connecting pipe further comprises a first through-hole, and the first pressure gauge is connected to the first through-hole to measure the gas pressure of the first connecting pipe.

According to one example of the present disclosure, the second pressure gauge is positioned on the inner wall of the throat of the flexible pipe.

According to one example of the present disclosure, the pipe wall of the throat of the flexible pipe further comprises a second through-hole, and the second pressure gauge is connected to the second through-hole to measure the gas pressure of the is throat of the flexible pipe.

According to one example of the present disclosure, the respirator is a full-face mask, a nasal mask or a nasal pillow mask.

### BRIEF DESCRIPTION OF THE DRAWINGS

For a more complete understanding of the present invention, and the advantages thereof, reference is now made to the following descriptions taken in conjunction with the accompanying drawings, in which:

FIG. 1A is a schematic view depicting a normal breathing condition of people when sleeping;

FIG. 1B is a schematic view depicting an upper airway resistance syndrome of people when sleeping;

FIG. 1C is a schematic view depicting obstructive sleep apnea (OSA) occurs when sleeping;

FIG. 2 shows a diagram using a conventional positive airway pressure apparatus (PAPA) to treat the OSA;

FIG. 3 is a curve graph of gas flow rate to gas pressure of a conventional gas flow detector, wherein the horizontal axis is gas flow rate (liter per minute, LPM), and the vertical axis is gas pressure (hPa);

FIG. 4A is a three-dimensional view of a conduit 400 according to an embodiment of the present disclosure;

FIG. 4B is a cross-sectional view of a conduit 400 according to an embodiment of the present disclosure;

FIG. 5 is a schematic view of a gas flow detector 500 according to an embodiment of the present disclosure;

FIG. 6A is a cross-sectional view of a flexible pipe 600 according to an embodiment of the present disclosure;

FIG. 6B is a sectional view of a flexible pipe 600 taken along A-A′ line of FIG. 6A;

FIG. 7 is a curve graph of gas flow rate to gas pressure, wherein the horizontal axis is gas flow rate (LPM), and the vertical axis is gas pressure (hPa); and

FIG. 8 is a schematic view of a positive airway pressure apparatus (PAPA) 800 according to an embodiment of the present disclosure.

### DETAILED DESCRIPTION

The embodiments of the gas flow detector and a positive airway pressure apparatus (PAPA) containing the same of the present disclosure are discussed in detail below, but not limited the scope of the present disclosure. The same symbols or numbers are used to the same or similar portion in the drawings or the description. And the applications of the present disclosure are not limited by the following embodiments and examples which the person in the art can apply in the related field.

FIG. 4A is a three-dimensional view of a conduit 400 according to an embodiment of the present disclosure; and FIG. 4B is a cross-sectional view of a conduit 400 according to an embodiment of the present disclosure. In FIG. 4A, a conduit 400 comprises a flexible pipe 410, a first connecting pipe 420 and a second connecting pipe 430. In FIG. 4B, the flexible pipe 410 comprises a contraction portion 411, a throat 412 and an expanding portion 413. In which the throat 412 is sandwiched between the contraction portion 411 and the expanding portion 413, and the inner diameter of the throat 412 is smaller than the inner diameters of the contraction portion 411 and the expanding portion 413.

Thus, the flexible pipe 410 has the structure and properties of a Venturi tube, in which the gas pressure and the gas flow rate of the throat 412 and the contraction portion 411 have a relationship (Formula 1) as following:

$${P}_{2} - {P}_{1} = \frac{ρ}{2} ( {V}_{1}^{2} - {V}_{2}^{2} ) \qquad ( Formula 1 )$$

Wherein P₁ is the gas pressure (hPa) of the contraction portion 411,
P₂ is the gas pressure (hPa) of the throat 412,
V₁ is the gas flow rate (liter per minute, LPM) of the contraction portion 411,
V₂ is the gas flow rate (LPM) of the throat 412, and
ρ is a constant of proportionality.

According to one example of the present disclosure, the pipe wall of the throat 412 further comprises a second through-hole 414 providing a detecting point of gas pressure to detect the gas pressure of the flexible pipe 410, shown as FIG. 4A.

In FIG. 4B, the contraction portion 411, the throat 412 and the expanding portion 413 of the flexible pipe 410 may individually have a pipe wall in change of thickness. In that the flexible pipe 410 has the properties of flexible materials, the change in the thickness of the pipe wall of the portions in the flexible pipe 410 can have the effect on the gas pressure of the gas flow.

In particular, under a constant gas flow rate, if the thickness of the pipe wall becomes thicker, the impedance of the flow is greater. Conversely, if the thickness of the pipe wall is thinner, the impedance becomes smaller. Therefore, the best curve of the gas flow rate to the gas pressure is able to be regulated by varying the thickness of the pipe wall of the portions in the flexible pipe 410.

According to one example of the present disclosure, the Shore hardness of the flexible pipe 410 is in a range of HS10-HS90. According to one example of the present disclosure, the material of the flexible pipe 410 is an elastomer comprising silicone, rubber, polyurethane, latex or polytetrafluoroethylene (PTFE), but not to limit.

The first connecting pipe 420 is connected to the contraction portion 411 of the flexible pipe 410; and the second connecting pipe 430 is connected to the expanding portion 413 of the flexible pipe 410. In which, the materials of the first connecting pipe 420, the second connecting pipe 430 or the both comprise flexible materials or rigid materials. According to one example of the present disclosure, the materials of the first connecting pipe 420, the second connecting pipe 430 or the both are same as that of the flexible pipe 410. According to one example of the present disclosure, the pipe wall of the first connecting pipe 420 further comprises a first through-hole 421 providing a detecting point of gas pressure to detect the gas pressure of the first connecting pipe 420.

FIG. 5 is a schematic view of a gas flow detector 500 according to an embodiment of the present disclosure. In FIG. 5, the gas flow detector 500 comprises the mentioned conduit 400, a first pressure gauge 510 and a second pressure gauge 520. In which, the first pressure gauge 510 is connected to the first through-hole 421 to detect and read the gas pressure of the first connecting pipe 420. And the second pressure gauge 520 is connected to the second through-hole 414 to detect and read the gas pressure of the flexible pipe 410.

However, FIG. 5 is only an example of the present disclosure to demonstrate how the first and the second pressure gauges connected to the conduit and detecting the gas pressure of specific points in the conduit, but not to limit the present disclosure. According to another example of the present disclosure, the first pressure gauge is positioned on the inner wall of the first connecting pipe; and the second pressure gauge is positioned on the inner wall of the throat of the flexible pipe.

FIG. 6A is a cross-sectional view of a flexible pipe 600 according to an embodiment of the present disclosure; and FIG. 6B is a sectional view of a flexible pipe 600 taken along A-A′ line of FIG. 6A. In FIG. 6A, the flexible pipe 600 comprises a contraction portion 610, a throat 620, an expanding portion 630 and a honeycomb structure 640, wherein the honeycomb structure 640 is positioned in the throat 620. FIG. 6B is shown that, due to the honeycomb structure 640, the throat 620 has a cross-sectional in a honeycomb shape. In FIG. 6A-6B, a honeycomb structure 640 is positioned in a Venturi tube to contract the cross-sectional area of the throat 620, so as to increase the pressure difference between the throat 620 and the contraction portion 610.

FIG. 7 is a curve graph of gas flow rate to gas pressure, wherein the horizontal axis is gas flow rate (liter per minute, LPM), and the vertical axis is gas pressure (hPa). In FIG. 7, a curve 710 is the relationship of gas flow rate to gas pressure of the conduit made of a rigid material; and a curve 720 is the relationship of gas flow rate to gas pressure of the conduit according to one example of the present disclosure. Compared to the curve 710, the slope of the curve 720 is larger at low gas flow rate (20-80 LPM), so as to have higher resolution. Otherwise, the slope of the curve 720 is smaller at high gas flow rate (>100 LPM), so as to keep the gas pressure of the conduit in the detectable range of a detector. Thus, according to one example of the present disclosure, the gas flow detector may have larger detectable range of gas flow rate, under a fixed detection limitation of gas pressure.

FIG. 8 is a schematic view of a positive airway pressure apparatus (PAPA) 800 according to an embodiment of the present disclosure. The PAPA 800 comprises a flow generator 810, the gas flow detector 500 shown as FIG. 5, a respirator 820 and a control circuit 830.

The flow generator 810 is configured to generate a positive pressure gas flow 811. The first connecting pipe 420 of the conduit 400 in the gas flow detector 500, refer to FIG. 5, is connected to the flow generator 810, so as to introduce the positive pressure gas flow 811 into the flexible pipe 410. The respirator 820 is connected to the second connecting pipe 430 of the conduit 400 in the gas flow detector 500, so as to introduce the positive pressure gas flow 811 into the airway of a person 840 under treatment, and the airway is kept in positive pressure. In which, the respiratory is a respiration auxiliary device, such as a full-face mask, a nasal mask or a nasal pillow mask.

The control circuit 830 is electrically connected to the flow generator 810 and the first pressure gauge 510 and the second pressure gauge 520 of the gas flow detector 500. After the first pressure gauge 510 and the second pressure gauge 520 respectively detect and read out the gas pressure of the first connecting pipe 420 and the flexible pipe 410, the control circuit 830 may regulate the positive pressure gas flow 811 output from the flow generator 810, so as to satisfy the necessary of the user 840.

Although embodiments of the present disclosure and their advantages have been described in detail, they are not used to limit the present disclosure. It should be understood that various changes, substitutions and alterations can be made herein without departing from the spirit and scope of the present disclosure. Therefore, the protecting scope of the present disclosure should be defined as the following claims.

What is claimed is:

1. A gas flow detector, comprising:
a conduit comprising:
a flexible pipe having a contraction portion, a throat and an expanding portion, wherein the throat is sandwiched between the contraction portion and the expanding portion, and the inner diameter of the throat is smaller than the inner diameters of the contraction portion and the expanding portion;
a first connecting pipe connected to the contraction portion of the flexible pipe; and
a second connecting pipe connected to the expanding portion of the flexible pipe;
a first pressure gauge configured to measure the gas pressure of the first connecting pipe; and
a second pressure gauge configured to measure the gas pressure of the throat of the flexible pipe,
wherein the gas flow rates of the first connecting pipe and the throat of the flexible pipe are calculated based on the difference of the gas pressures read from the first and the second pressure gauges.

2. The gas flow detector of claim 1, wherein the Shore hardness of the flexible pipe is in a range of HS10-HS90.

3. The gas flow detector of claim 1, wherein the material of the flexible pipe is an elastomer comprising silicone, rubber, polyurethane, latex or polytetrafluoroethylene (PTFE).

4. The gas flow detector of claim 1, wherein the material of the first connecting pipe, the second connecting pipe or the both comprise a flexible material or a rigid material.

5. The gas flow detector of claim 4, wherein the material of the first connecting pipe, the second connecting pipe or the both are same as that of the flexible pipe.

6. The gas flow detector of claim 4, wherein the contraction portion, the throat and the expanding portion of the flexible pipe individually have a pipe wall in change of thickness.

7. The gas flow detector of claim 1, further comprising a honeycomb structure positioned in the throat of the flexible pipe, wherein the throat has a cross-section in a honeycomb shape.

8. The gas flow detector of claim 1, wherein the first pressure gauge is positioned on the inner wall of the first connecting pipe.

9. The gas flow detector of claim 1, wherein the pipe wall of the first connecting pipe further comprises a first through-hole, and the first pressure gauge is connected to the first through-hole to measure the gas pressure of the first connecting pipe.

10. The gas flow detector of claim 1, wherein the second pressure gauge is positioned on the inner wall of the throat of the flexible pipe.

11. The gas flow detector of claim 1, wherein the pipe wall of the throat of the flexible pipe further comprises a second through-hole, and the second pressure gauge is connected to the second through-hole to measure the gas pressure of the throat of the flexible pipe.

12. A positive airway pressure apparatus (PAPA), comprising:
a flow generator configured to generate a positive pressure gas flow;
a gas flow detector as claim 1, wherein the first connecting pipe of the conduit is connected to the flow generator, so as to introduce the positive pressure gas flow into the flexible pipe;
a respirator connected to the second connecting pipe of the conduit of the gas flow detector, so as to introduce the positive pressure gas flow into the airway of a person, and keep positive pressure in the airway;
a control circuit respectively electrically connected to the flow generator and the first and the second pressure gauge of the gas flow detector, wherein the first and the second pressure gauge read the pressure values, such that the positive pressure gas flow of the flow generator is regulated.

13. The PAPA of claim 12, wherein the Shore hardness of the flexible pipe is in a range of HS10-HS90.

14. The PAPA of claim 12, wherein the material of the flexible pipe is an elastomer comprising silicone, rubber, polyurethane, latex or polytetrafluoroethylene (PTFE).

15. The PAPA of claim 12, wherein the material of the first connecting pipe, the second connecting pipe or the combination comprises a flexible material or a rigid material.

16. The PAPA of claim 15, wherein the material of the first connecting pipe, the second connecting pipe or the combination are same as the flexible pipe.

17. The PAPA of claim 12, further comprising a honeycomb structure positioned in the throat of the flexible pipe, wherein the throat has a cross-section in a honeycomb shape.

18. The PAPA of claim 12, wherein the first pressure gauge is positioned on the inner wall of the first connecting pipe.

19. The PAPA of claim 12, wherein the pipe wall of the first connecting pipe further comprises a first through-hole, and the first pressure gauge is connected to the first through-hole to measure the gas pressure of the first connecting pipe.

20. The PAPA of claim 12, wherein the second pressure gauge is positioned on the inner wall of the throat of the flexible pipe.

21. The PAPA of claim 12, wherein the pipe wall of the throat of the flexible pipe further comprises a second through-hole, and the second pressure gauge is connected to the second through-hole to measure the gas pressure of the throat of the flexible pipe.
